(19) United States
(12) Patent Application Publication
(10) Pub. No.: US 2012/0208149 A1
(43) Pub. Date: Aug. 16, 2012

(54) DENTAL COMPOSITE MATERIAL AND APPLICATIONS THEREOF

(75) Inventors: Bor-Shiunn LEE, Taipei (TW); Fa-Jen Wang, Taipei (TW); Chi-An Dai, Taipei (TW); Chien-Liang Liu, Taipei (TW); Da-Ming Wang, Taipei (TW); Yi-Chen Kuo, Taipei (TW)

(73) Assignee: National Taiwan University, Taipei (TW)

(21) Appl. No.: 13/092,315

(22) Filed: Apr. 22, 2011

(30) Foreign Application Priority Data
Feb. 11, 2011 (TW) 100104589

(51) Int. Cl. A61C 5/02; A61K 6/083

(52) U.S. Cl. 433/224; 523/116

(57) ABSTRACT

A dental composite material comprises a composite resin and an adjuvant with a concentration of more than 10% by weight of the dental composite material, wherein the adjuvant comprises more than 20% 1,6-Hexanediol Diacrylate (HDDA) by weight of the adjuvant, more than 20% Tripropylene Glycol Diacrylate (TPGDA) by weight of the adjuvant and a component selected from the group consisting of Ethyl Hexyl Acrylate (EHA), Isobornyl Acrylate (IBOA), 2 Hydroxy 3 Phenoxypropyl Acrylate (DM120) and the arbitrary compositions thereof with a concentration of more than 20% by weight of the adjuvant.

### BACKGROUND

1. Technical Field

Present invention is related to a dental composite material and the applications thereof, more particularly related to a dental composite material consisting of a composite resin and an adjuvant and the applications thereof.

2. Description of the Related Art

Root canal treated teeth with insufficient coronal structure generally require radicular posts for crown restoration. Traditionally, cast metal posts and cores covered by porcelain fused to metal crowns are the choice for anterior teeth restoration.

Recently fiber posts which can provide an even stress distribution along the teeth have gradually become accepted to replace cast metal posts, because the esthetic outcomes for anterior teeth are more pleasing and the elastic moduli of these posts are approximate to that of dentin.

Fiber posts commonly consist of a high proportion of reinforcing fibers embedded in epoxy resins with a highly cross-linked structure. The use of adhesive resin cements in luting fiber posts is recommended to improve the retention, reduce micro-leakage, and elevate resistance to tooth fracture. The bonding mechanism of resin cement to root canal dentin is basically micromechanical from hybridization of the demineralized surface and resin tag formation.

However, since the adhesion between resin cement and dentin was considered weaker than the adhesion between resin cement and fiber posts, thus applying resin cement to luting fiber posts with root canal dentin is still not reliable. In addition, the complex environment of the root canal system including the problems of using endodontic sealers, irrigating solutions, and medicaments may further deteriorate the bond strength among resin cement, the fiber post and the root canal dentin. Furthermore, in comparison with coronal dentin, root canal dentin has a limited visibility, irregular structure in the apical region, and a high configuration factor, and these characteristics may make the problem of the bond strength worse during practical dental operations.

Therefore, it is necessary to provide an improved dental composite material and the applications thereof to enhance the reliability of the bond strength among resin cement, the fiber post and the root canal dentin, so as to elevate resistance of the restored tooth.

### BRIEF SUMMARY

One aspect of the present invention is to provide a dental composite material, wherein the dental composite material comprises a composite resin and an adjuvant with a concentration of more than 10% by weight of the dental composite material, and the adjuvant comprises more than 20% 1,6-Hexanediol Diacrylate (HDDA) by weight of the adjuvant, more than 20% Tripropylene Glycol Diacrylate (TPGDA) by weight of the adjuvant and a component selected from the group consisting of Ethyl Hexyl Acrylate (EHA), Isobornyl Acrylate (IBOA), 2 Hydroxy 3 Phenoxypropyl Acrylate (DM120) and the arbitrary compositions thereof with a concentration of more than 20% by weight of the adjuvant.

In some embodiments of the present invention, the adjuvant comprises 25% HDDA, 25% TPGDA and 50% IBOA by weight of the adjuvant.

In some embodiments of the present invention, the adjuvant comprises 25% HDDA, 25% TPGDA and 50% DM120 by weight of the adjuvant.

In some embodiments of the present invention, the adjuvant comprises 25% HDDA, 25% TPGDA and 50% EHA by weight of the adjuvant.

In some embodiments of the present invention, the adjuvant comprises 25% HDDA, 25% TPGDA, 25% EHA and 25% IBOA by weight of the adjuvant.

In some embodiments of the present invention, the composite resin comprises 2,2-bis-4-2-hydroxy-3-methacryloxypropoxy-propane and triethylene-glycol-dimethacrylate (TEGDMA).

In some embodiments of the present invention, the dental composite material further comprises photoactives with a concentration of less than 2% by weight of the dental composite material. In some preferred embodiments, the photoactives comprises camphorquinone (CQ), ethyl 4-dimethylaminobenzoate (EDMAB) and Benzoyl peroxide (BPO).

Another aspect of the present invention is to provide a method for restoring tooth after root canal treatment comprising steps as follows: A dental composite material is firstly filled into a root canal, wherein the dental composite material comprises a composite resin and an adjuvant with a concentration of more than 10% by weight of the dental composite material, and the adjuvant comprises more than 20% HDDA by weight of the adjuvant, more than 20% TPGDA by weight of the adjuvant and a component selected from the group consisting of EHA, IBOA, DM120 and the arbitrary compositions thereof with a concentration of more than 20% by weight of the adjuvant. Subsequently, at least one root canal post is disposed in the root canal, and the dental composite material is then cured to fasten the root canal post.

In some embodiments of the present invention, the root canal post is a cast metal post or a fiber post.

In accordance with the aforementioned embodiments, the present invention is to provide a dental composite material having HDDA, TPGDA and a component selected from the group consisting of EHA, IBOA, DM120 and the arbitrary compositions thereof serving as adjuvant of dental cement or dental adhesive for cementation of root canal posts, especially for tooth restoration after root canal treatment, to enhance the reliability of the bond strength among the dental cement, fiber posts and root canal dentin, so as to elevate resistance of the restored tooth.

In order to make the aforementioned and other objects, features and advantages of the present invention comprehensible, preferred embodiments accompanied with figures are described in detail below. Other objectives, features and advantages of the present invention will be further understood from the further technological features disclosed by the embodiments of the present invention wherein there are shown and described preferred embodiments of this invention, simply by way of illustration of modes best suited to carry out the invention.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 illustrates the push-out bond strength histogram of the dental specimens in accordance with one embodiment of the present invention.

FIGS. 2A to 2E are photographs of the dental specimens taken by a scanning electron microscopy (SEM) after the push-out bond test is conducted, in accordance with one preferred embodiment of present invention.

FIG. 3 illustrates the asymmetric double cantilever beam apparatus for measuring the fracture toughness in accordance with one embodiment of the present invention.

FIG. 4 illustrates the histogram of the interfacial fracture toughness measured by the asymmetric double cantilever beam method shown in the FIG. 3 in accordance with one embodiment of the preset invention.

FIG. 5 shows the FTIR spectra of the root dentin half specimens provided by the fracture toughness test shown in FIG. 3.

FIG. 6 illustrates XPS spectra of the root dentin half specimens provided by the fracture toughness test shown in FIG. 3.

### DETAILED DESCRIPTION

The object of the present invention is to provide a dental composite material used to enhance the reliability of the bond strength among dental cement, fiber posts and root canal dentin, so as to elevate resistance of the restored tooth. Various inspection or tests including a push-out-bond test and a quantitative-fracture-toughness test are conducted to evaluate the adhesion among the present dental composite material, fiber posts and root canal dentin and to validate whether the dental composite material provided by the present invention can enhance the reliability of the bond strength between fiber posts and root canal dentin and elevate resistance of restored tooth.

In the embodiments of the present invention, the dental composite material at least comprises composite resin and an adjuvant, and preferably further comprises photoactives. In some embodiments of the present invention, the adjuvant has a concentration substantially about 10% by weight of the dental composite material; and the content of the photoreactives is less than 2% by weight of the dental composite material. The material consisting of the composite resin comprises 2,2-bis-4-2-hydroxy-3-methacryloxypropoxy-propane and TEGDMA; and the material consisting of the photoactives comprises CQ, EDMAB and BPO.

The material consisting of the adjuvant comprises HDDA, TPGDA and a component selected from the group consisting of EHA, IBOA, DM120 and the arbitrary compositions thereof, wherein HDDA and TPGDA respectively have more than 20% of the adjuvant by weight, and the component selected from the group consisting of EHA, IBOA, DM120 and the arbitrary combination thereof has a concentration of more than 20% by weight of the adjuvant. To describe the make, use and applications of the present invention, several preferred embodiments with detail contents of the dental composite material are described in detail as following:

### Embodiment I

A dental composite material consisting of 88.75% composite resin, 10% adjuvant and 1.25% photoactives by weight of the dental composite material is provided. Wherein the composite resin consists of 2,2-bis-4-2-hydroxy-3-methacryloxypropoxy-propane and TEGDMA with a molar ratio substantially about 1:1. The adjuvant consists of 25% HDDA, 25% TPGDA and 50% IBOA by weight of the adjuvant. The photoactives comprises 0.25% CQ, 0.5% EDMAB and 0.5% BPO by weight of the dental composite material.

### Embodiment II

A dental composite material consisting of 88.75% composite resin, 10% adjuvant and 1.25% photoactives by weight of the dental composite material is provided. Wherein the composite resin consists of 2,2-bis-4-2-hydroxy-3-methacryloxypropoxy-propane and TEGDMA with a molar ratio substantially about 1:1. The adjuvant consists of 25% HDDA, 25% TPGDA and 50% DM120 by weight of the adjuvant. The photoactives comprises 0.25% CQ, 0.5% EDMAB and 0.5% DM120 by weight of the dental composite material.

### Embodiment III

A dental composite material consisting of 88.75% composite resin, 10% adjuvant and 1.25% photoactives by weight of the dental composite material is provided. Wherein the composite resin consists of 2,2-bis-4-2-hydroxy-3-methacryloxypropoxy-propane and TEGDMA with a molar ratio substantially about 1:1. The adjuvant consists of 25% HDDA, 25% TPGDA and 50% EHA by weight of the adjuvant. The photoactives comprises 0.25% CQ, 0.5% EDMAB and 0.5% DM120 by weight of the dental composite material.

### Embodiment IV

A dental composite material consisting of 88.75% composite resin, 10% adjuvant and 1.25% photoactives by weight of the dental composite material is provided. Wherein the composite resin consists of 2,2-bis-4-2-hydroxy-3-methacryloxypropoxy-propane and TEGDMA with a molar ratio substantially about 1:1. The adjuvant consists of 25% HDDA, 25% TPGDA, 25% EHA and 25% IBOA by weight of the adjuvant. The photoactives comprises 0.25% CQ, 0.5% EDMAB and 0.5% DM120 by weight of the dental composite material.

Subsequently, a push-out-bond test and a quantitative-fracture-toughness test are conducted, and a scanning electron microscopy (SEM), an attenuated total reflection-Fourier transform infrared spectroscopy (ATR-FTIR) and an X-ray Photoelectron Spectroscopy (XPS) are utilized to investigate and characterize the morphology and the microstructure of the fractured interfaces between the present dental composite material and the root canal dentin, to evaluate the cohesive failure resulted from the quantitative-fracture-toughness test.

(1) Push-Out-Bond test

Groups of dental specimens 1-4 are selected to associated with the 4 kinds of dental composite materials set forth in the embodiments □-□, wherein each of the dental specimens is prepared to form a 1.8 mm-diameter hole at least 1 mm from the edge of specimen and the hole is centrally prepared in each root segment preceding the push-out-bond test.

The 1.8-mm diameter holes in the dental specimens are etched for 15 seconds with an etchant comprising 37.5% phosphoric acid, rinsed thoroughly, and air dried. In some embodiments of the present invention, the etchant preferably is Kerr Gel Etchant provided by Kerr Corporation (CA, USA). In addition, a 1.8-mm diameter hole is prepared and etched in each dental specimen of a 5th group serving as a control.

An etch-and-rinse 2-step adhesive system, such as Optibond Solo Plus provided by Kerr Corporation (CA, USA), is then applied for 15 seconds and the excessive adhesive is removed with absorbent paper points. Subsequently, the top and bottom surfaces of the dental specimens are light-cured for 10 seconds with a light curing machine (Dentsply, provided by SmartLite Corporation, PA, USA) at an intensity of 800 mW/cm². Then, the canals of the groups 1-4 dental specimens are respectively filled with the 4 kinds of dental composite materials set forth in the embodiments □-□, and are light-cured again for an additional 40 seconds at top and bottom surfaces.

The 5th group control specimens are otherwise filled with PANAVIA F 2.0 (provided by Kuraray Corporation) and treated according to the manufacturer's instructions. For the purpose of clearly describing the following embodiment, the groups 1-4 dental specimens that are respectively filled with the 4 kinds of dental composite materials set forth in the embodiments □-□ and the Panavia F 2.0™ are referred as IBOA ’ DM120’ EHA ’ IBOA+EHA and PANAVIA F 2.0 thereinafter.

Each root segment is serially sectioned into 1 mm-thick root slices using a high-speed diamond wafering blade (e.g. Isomet 2000 Precision High-Speed Saw; Buehler Ltd), and the thickness is verified using an electronic vernier (e.g. CD-I0CX™; Mitutoyo Co Ltd, Tokyo, Japan). Fifty root slices of the groups 1-4 dental specimens are randomly divided into 5 groups with 10 root slices in each group. Next, a cylindrical carbon steel rod 1.7 mm in diameter is used as a plunger, whereby a 1.75 mm-diameter hole and a 1.85 mm-diameter hole are centrally positioned at the upper and lower surfaces of each slices, respectively. The push-out bond strength is calculated using the following equation:

Push-out bond strength(MPa)=Force/(π×Diameter×Thickness)

The testing results are analyzed using two-tailed analysis of variance (ANOVA), with a significance level of 0.05 and follow-up comparison between the groups is made using Fisher's multiple comparison tests.

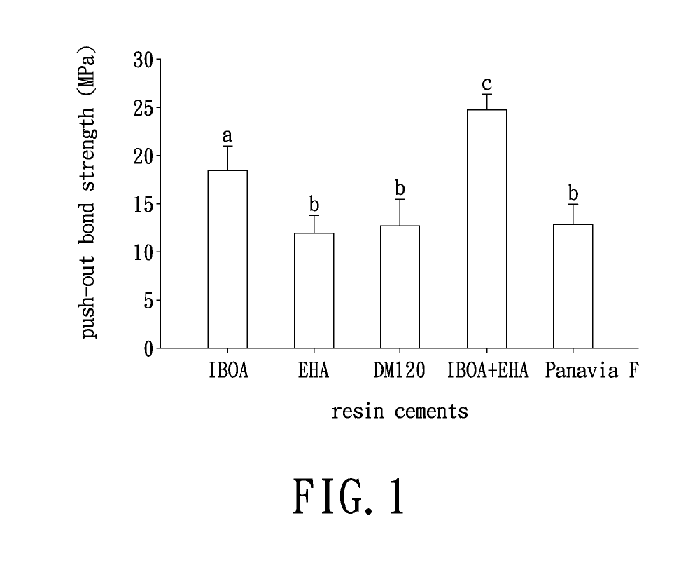

FIG. 1 illustrates the push-out bond strength histogram of the dental specimens that are respectively filled with the 4 kinds of dental composite materials set forth in the embodiments I-IV and the PANAVIA F 2.0 in accordance with one embodiment of the present invention. The X-axis of the histogram indicates the resin cements (including the dental composite materials and the control the PANAVIA F 2.0) respectively filled in the specimens, the Y-axis indicates the push-out bond strength (MPa), and different letters (such as a, b and c) represent significant difference.

The push-out bond test revealed that IBOA+EHA specimens exhibit the highest push-out bond strength followed by IBOA specimens, and both of them has higher push-out bond strength than that of the Panavia F 2.0. Even EHA and DM120 specimens with lower bond strengths are not significantly different from that of PANAVIA F 2.0. It demonstrates that the adhesive performance of the dental composite materials set forth in the embodiments □-□ is similar to that of the control PANAVIA F 2.0.

(2) SEM Observation

After the push-out bond test the morphology and microstructure of the fractured dental specimens are observed under SEM observation. Preceding the observation, the dental specimens are immersed in 2.5% cold glutaraldehyde in 0.1 mol/L cacodylate buffer at pH 7.4 for 8 hours. All dental specimens are then serially dehydrated in graded ethanol solutions (50%, 60%, 70%, 80%, 90%, 95%, and 100% ethanol) at 45 minutes intervals, mounted on aluminum stubs, and sputter-coated with carbon. The treated dental specimens are then observed under a Philips 515 (Mohawk Corporation, NJ, US) SEM, wherein the electron beams maintained at 2×10⁻¹⁰ amp are used, X-ray intensities in counts per second are recorded and the accelerating voltage is 15 kV.

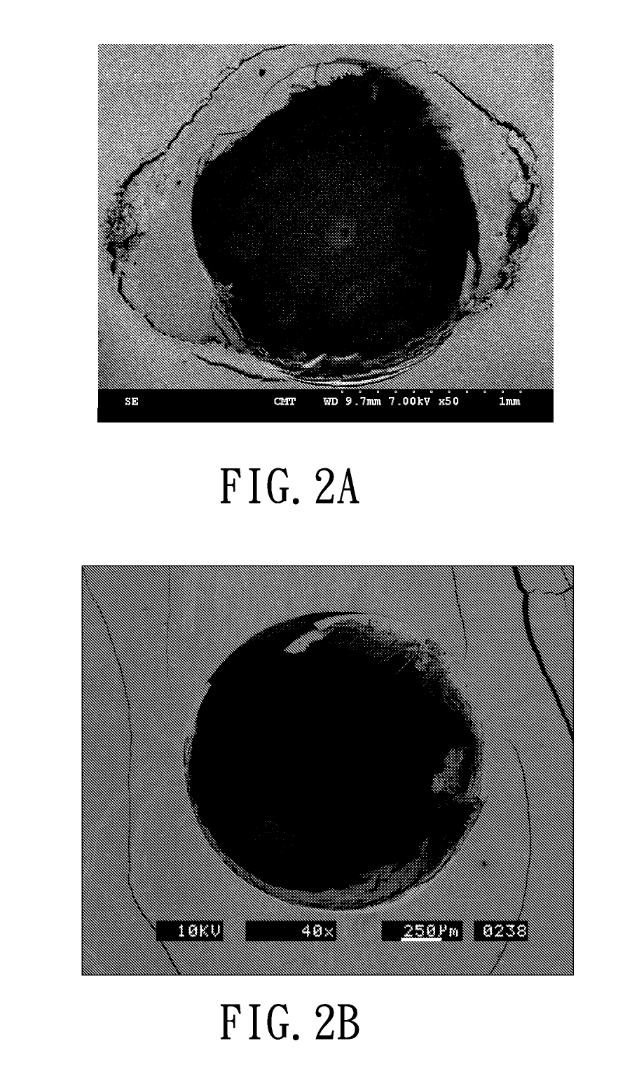

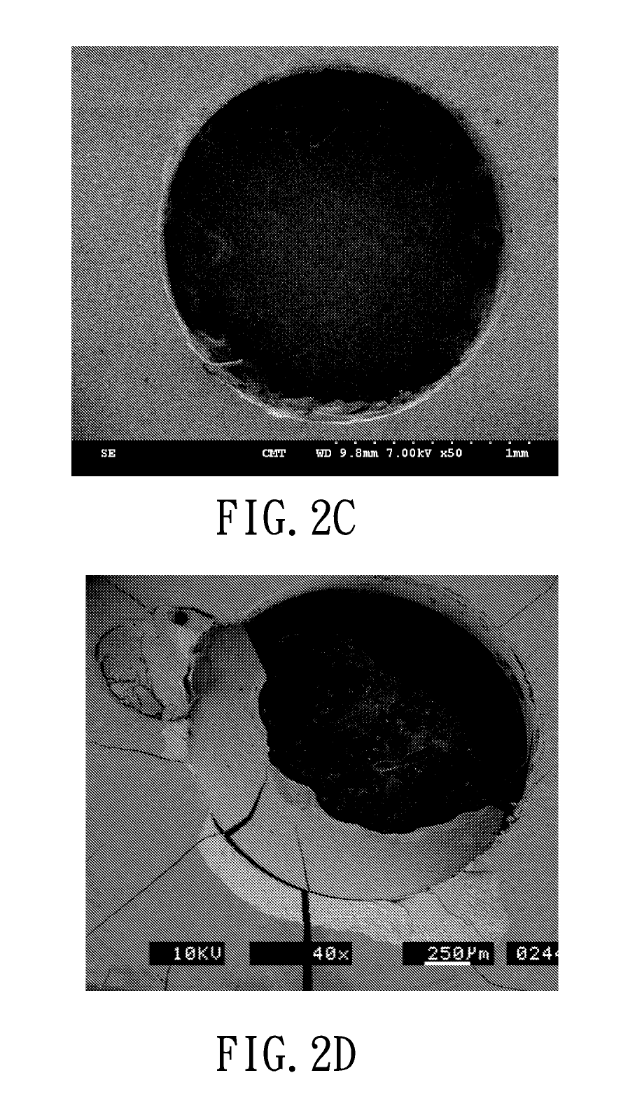

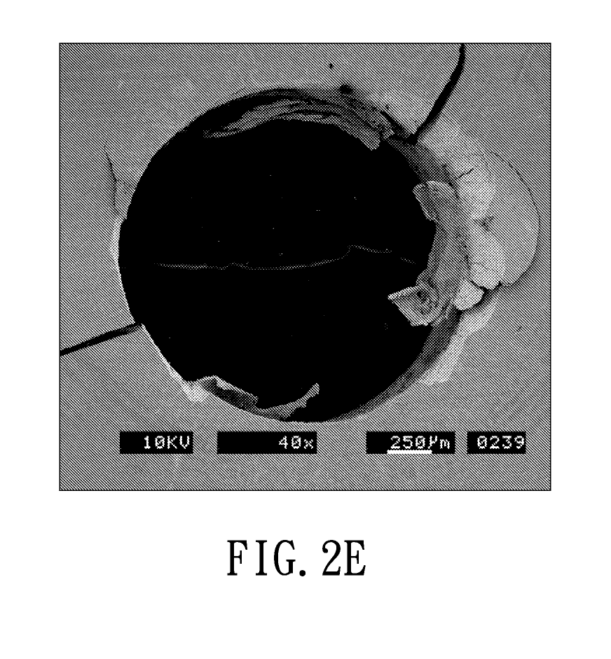

FIGS. 2A to 2E are photographs of the dental specimens taken by a SEM after the push-out bond test is conducted, in accordance with one preferred embodiment of present invention. FIG. 2A is a SEM photograph of the dental specimens filled with IBOA; FIG. 2B is a SEM photograph of the dental specimens filled with DM120; FIG. 2C is a SEM photograph of the dental specimens filled with EHA; FIG. 2D is a SEM photograph of the dental specimens filled with IBOA+EHA; and FIG. 2E is a SEM photograph of the dental specimens filled with PANAVIA F 2.0 taken after the push-out bond test is conducted.

FIGS. 2A and 2D reveal that a greater amount of resin cement remnants is left on the root dentin of the IBOA and IBOA+EHA specimens than that left on the other three groups. It means that a greater bonding may constitute between the root dentin and the dental composite material (e.g. IBOA and IBOA+EHA) than that between the root dentin and the traditional resin cement (e.g. the control specimens PANAVIA F 2.0).

FIGS. 2B and 2C reveal that EHA and DM120 specimens left less resin cement remnants than that of IBOA and IBOA+EHA, but the specimens of EHA and DM120 are still has morphology not significantly different from that of PANAVIA F 2.0.

(3) Quantitative-Fracture-Toughness Test

Fifty dental specimens from root dentin sized of 1.5 mm×3 mm×25 mm (thickness×width×length) are prepared and allotted in 5 groups. The 4 kinds of dental composite materials set forth in the embodiments I-IV and the control PANAVIA F 2.0 (5th Group) with a size of 1.5 mm×3 mm×25 mm (thickness×width×length) are adhered onto the dental specimens by the etch-and-rinse 2-step adhesive system identical to that used in the push-out bond test. An asymmetric double cantilever beam (ADCB) method is then applied to measure the interfacial fracture toughness (thereinafter referred as “Gc”) between the dental composite materials and the dental specimens.

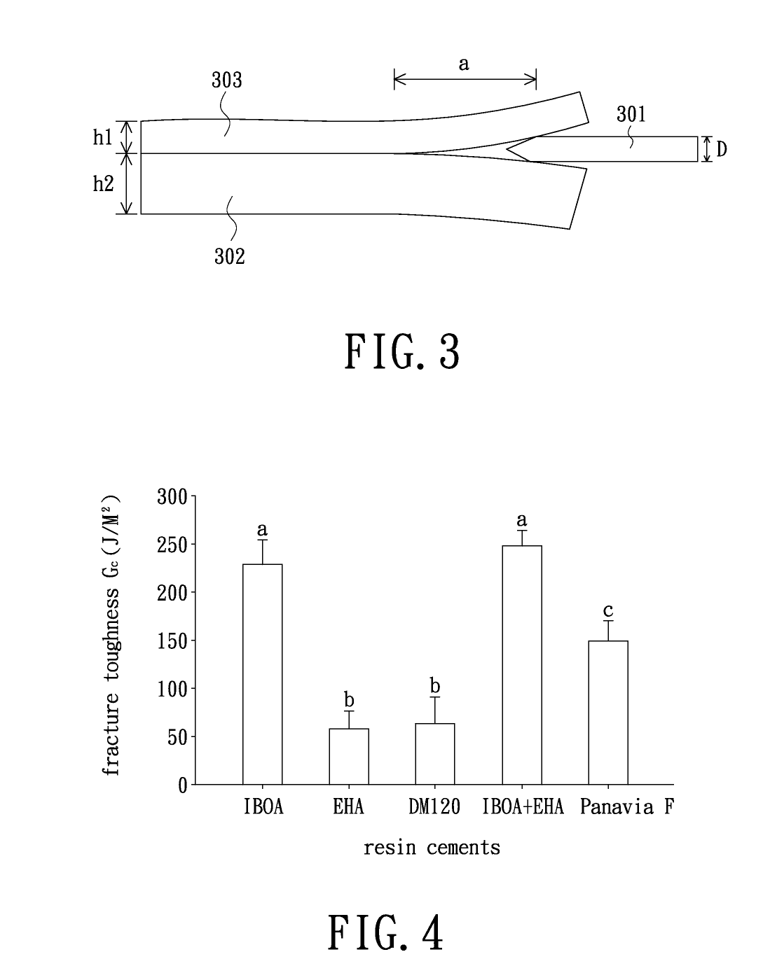

FIG. 3 illustrates the asymmetric double cantilever beam apparatus for measuring the fracture toughness in accordance with one embodiment of the present invention. A razor blade 301 of known thickness D driven by a servo motor at a constant speed (5×10⁻⁶ m/s) is inserted into the interface between a dental composite material 302 and the root dentin 303. A crack is then initiated ahead of the razor edge to make the dental composite material 302 separated from the root dentin 303, and steady state crack propagation is established after several minutes. The fracture toughness of the interface (G_{c}, J/m²) for a small crack in a bi-material each with finite elasticity could be measured based on Kanninen's calculation with the following equation:

$${G}_{c} = \frac{3 {D}^{2}}{8 {a}^{4}} ( {E}_{1} {E}_{2} {h}_{1}^{3} {h}_{2}^{3} ) \frac{( {C}_{1}^{2} {E}_{2} {h}_{2}^{3} + {C}_{2}^{2} {E}_{1} {h}_{1}^{3} )}{{( {C}_{1}^{3} {E}_{2} {h}_{2}^{3} + {C}_{2}^{3} {E}_{1} {h}_{1}^{3} )}^{2}}$$

Where C₁=1+0.64h₁/a and C₂=1+0.64h₂/a. “E₁” and “E₂” are Young's moduli; “a” is the length of the crack; and “h₁” and “h₂” are the thickness of the root dentin 303 and the dental composite material 302, respectively. The testing results are analyzed using two-tailed analysis of variance (ANOVA), with a significance level of 0.05 and follow-up comparison between the groups is made using Fisher's multiple comparison tests.

FIG. 4 illustrates the histogram of the interfacial fracture toughness measured by the asymmetric double cantilever beam method shown in the FIG. 3 in accordance with one embodiment of the preset invention.

FIG. 4 reveals that IBOA and IBOA+EHA specimens have greater G_{c} than that of PANAVIA F 2.0.

(4) SEM-EDX Analysis

After the fracture toughness test, the specimens are separated into two groups (root dentin half and dental composite material half). The root dentin half is examined with SEM-energy dispersive X-ray spectroscopy (SEM-EDX) to determine the atomic ratio of the dentin half. The following table shows the SEM-EDX results for the atomic ratio of the root dentin half.

TABLE
SEM-EDX results of atomic ratio (%) of fractured surfaces.

| | C | Ca | N | O | Na | Si | P | S |
|---|---|---|---|---|---|---|---|---|
| Dentin | 23.6 | 10.6 | 13.2 | 43.7 | 0.6 | 0.1 | 8.2 | 0.2 |
| Dentin-Optibond | 42.5 | 0.5 | 11.9 | 37.8 | 0.3 | 5.9 | 0.9 | 0.2 |
| Dentin-IBOA | 47.2 | 1.9 | 20.0 | 27.6 | 0.2 | 0 | 2.5 | 0.7 |
| Dentin-EHA | 47.2 | 0.9 | 25.7 | 23.7 | 0.2 | 0.5 | 1.2 | 0.6 |
| Dentin-DMI20 | 45.8 | 2.4 | 20.1 | 27.7 | 0.3 | 0 | 3.1 | 0.6 |
| Dentin-IBOA + EHA | 56.1 | 0.3 | 16.2 | 25.7 | 0 | 0 | 1.1 | 0.6 |
| Dentin-Panavia F 2.0 | 46.8 | 1.4 | 20.1 | 27.3 | 0.7 | 0.9 | 2.5 | 0.5 |

The root dentin without any treatment having atomic ratios of C (23.6%) and Ca (10.6%) is different from that of the control specimens PANAVIA F 2.0 with the atomic ratios of C and Ca of 46.8% and 1.4%, and also different from that of another control group, of which the root dentin specimens are adhered with the commercial cement, Optibond Solo Plus (thereinafter referred as “Optibond”), with the atomic ratios of C and Ca are 42.5% and 0.5%. Because there still remains a certain amount of PANAVIA F 2.0 or the Optibond residue on the surface of the root dentin after the fracture toughness test is conducted, it reveals that covalent bonds may be formed between the root dentin and the control cements.

In comparison with the present 4 kinds of dental composite materials set forth in the embodiments □-□, the IBOA+EHA shows a C ratio of 56.1% apparently higher than that of root dentin without any treatment. The other specimens IBOA, EHA, DMI20 and Panavia F 2.0 show similar C ratio to Optibond. It reveals that after the fracture toughness test, there still remains a certain amount of the dental composite materials on the surface of the root dentin; and the strength of the covalent bonds formed between the root dentin and the dental composite materials are substantially the same to or even greater than that of the current commercial cements (PANAVIA F 2.0 or the Optibond).

(5) ATR-FTIR Analysis

After the fracture toughness test, the fractured surfaces of the root dentin/the dental composite material samples are studied using an ATR-FTIR (FTIR-4200, provided by Jasco International Co., Ltd., Tokyo, Japan) to determine the molecular structure of the fractured surfaces of the root dentin/the dental composite material, wherein the FTIR spectra are recorded by pressing the samples against a ZnSe ATR crystal with slow scan and a normal slit width and the wave number ranges of 4000-400 cm⁻¹ to evaluate the functional groups. To the purpose of making a reference, pure EHA, IBOA, DM120 and PANAVIA F 2.0 cements are also studied by the ATR-FTIR.

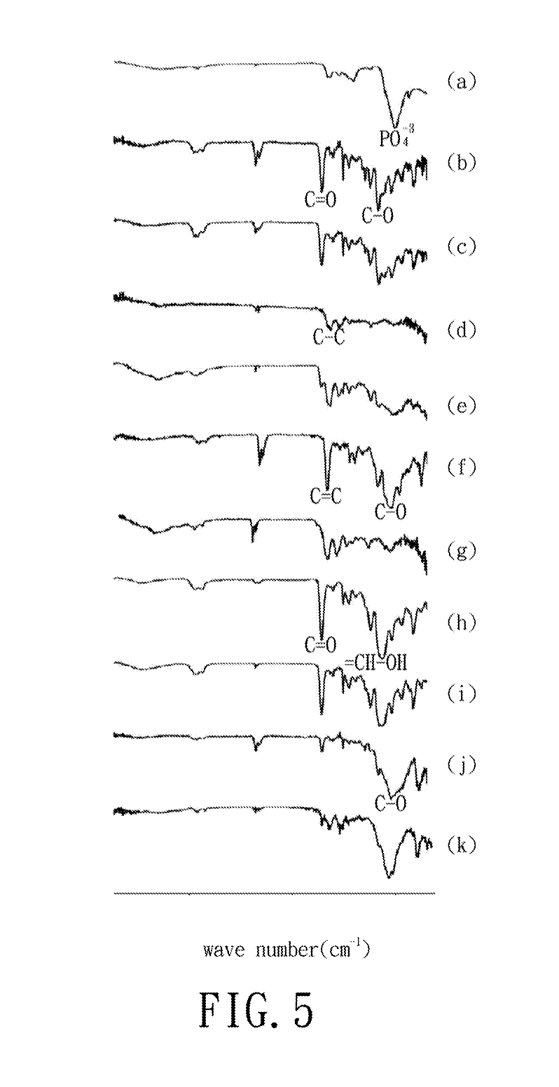

FIG. 5 shows the FTIR spectra of the root dentin half specimens provided by the fracture toughness test shown in FIG. 3. The X-axis of the FTIR spectra indicates wave number, the curve lines respectively represent the spectra of (a) the root dentin without treatment, (b) the pure IBOA cement (c) the fractured surfaces of the root dentin/IBOA, (d) the pure EHA cement, (e) the fractured surfaces of the root dentin/EHA, (f) the pure DM120 cement, (g) the fractured surfaces of the root dentin/DMI20, (h) the pure IBOA+EHA cement, (i) the fractured surfaces of the root dentin/IBOA+EHA, (j) the pure PANAVIA F 2.0 cement, and (k) the fractured surfaces of the root dentin/PANAVIA F 2.0.

The most intense bands associated with the PO₄³ vibrations, the anti-symmetric stretching mode ranges at 1100-1000 cm⁻¹, may characterize the presence of dentin; the strong bands due to the interaction of ester C—O stretching vibration with the C—C vibration occurred in the range of 1300-1100 cm⁻¹ which could be observed in the spectra of (b), (c), (f), (g), (j) and (k); The ester C═O stretching vibrations at 1740-1705 cm⁻¹ could be identified in the spectra of (b),(c),(h),(i); the spectra of (a)-(g) exhibit signals of non-conjugated alkenes with a weak C═C stretching absorption band in the range 1680-1620 cm⁻¹.

The ATR-FTIR analyses reveals that the FTIR spectra of the dentin half specimens for the 4 kinds of dental composite materials set forth in the embodiments □-□ and the control specimens PANAVIA F 2.0 are similar to those of pure resin cement. In other words, there remains a certain amount of bonds of C—O, C—C, C═O and C═C formed on the surface of the dentin half specimens after the fracture toughness test is conducted. Accordingly, it can be figured out that the present dental composite materials have the adhesion substantially the same to or even greater than that of the current commercial cements.

(6) XPS Analysis

The fractured surfaces of the root dentin/the dental composite materials samples after fracture toughness test are further studied using XPS (Thermo Scientific, UK). XPS spectra are acquired using K-alpha XPS with monochromatic Aluminum (Al) Kα X-rays (beam energy=1486.6 eV). Survey spectra are collected over the range of 0-1100 eV. The X-ray source is operated at 210 W and the system operating pressure is 5×10⁻⁸ Torr. All spectra are referenced to the C is peak of the aliphatic carbon atoms, which is assigned a value of 284.6 eV. To the purpose of making a reference, pure EHA, IBOA, DM120 and PANAVIA F 2.0 cements are also studied by the XPS.

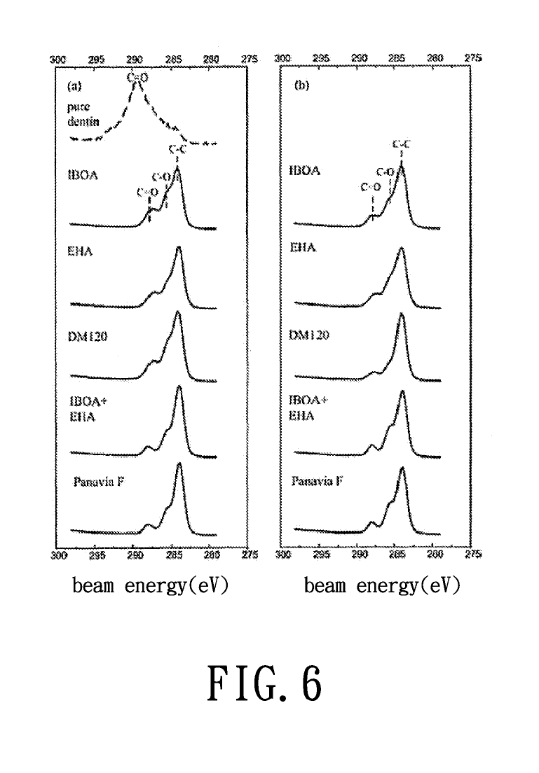

FIG. 6 illustrates XPS spectra of the root dentin half specimens provided by the fracture toughness test shown in FIG. 3, wherein the curve lines depicted in (a) indicate the XPS spectra of the pure EHA, IBOA, DM120 and PANAVIA F 2.0 cements and the curve lines depicted in (b) indicate the XPS spectra of the dentin half specimens of the fracture toughness test.

It is found that the XPS spectra of the dentin half specimens are similar to those of the pure resin cements. In addition, the XPS spectra for all samples are significantly different from that of the pure dentin sample. The above results indicate that a cohesive failure of the resin cement occurs on the resin cement near the dentin/resin cement interface. Furthermore, it can be conjectured that each of the dental composite materials set forth in the embodiments □-□ has an adhesion substantially the same to that of the current commercial cements.

In accordance with the aforementioned embodiments, the present invention is to provide a dental composite material having HDDA, TPGDA and a component selected from the group consisting of EHA, IBOA DM120 and the arbitrary compositions thereof serving as a dental cement or a dental adhesive for restoring tooth after root canal treatment to enhance the reliability of the bond strength among the dental cement, fiber posts and root canal dentin, so as to elevate resistance of the restored tooth. Those advantages and objects of the present invention can be verified by the aforementioned push-out-bond test, the quantitative-fracture-toughness test, the SEM Observation, the SEM-EDX analysis, the ATR-FTIR analysis, and the XPS analysis.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

1. A dental composite material, comprising:
a composite resin; and
an adjuvant with a concentration of more than 10% by weight of the dental composite material, wherein the adjuvant comprises more than 20% 1,6-Hexanediol Diacrylate (HDDA) with a concentration ranging from 20% to 25% by weight of the adjuvant, Tripropylene Glycol Diacrylate (TPGDA) with a concentration ranging from 20% to 25% by weight of the adjuvant and a component with a concentration ranging from 20% to 50% by weight of the adjuvant; and the component is selected from the group consisting of Ethyl Hexyl Acrylate (EHA), Isobornyl Acrylate (IBOA), 2 Hydroxy 3 Phenoxypropyl Acrylate (DM120) and the arbitrary compositions thereof.

2. The dental composite material as claimed in claim 1, wherein the adjuvant comprises 25% HDDA, 25% TPGDA and 50% IBOA by weight of the adjuvant.

3. The dental composite material as claimed in claim 1, wherein the adjuvant comprises 25% HDDA, 25% TPGDA and 50% 2 Hydroxy 3 Phenoxypropyl Acrylate by weight of the adjuvant.

4. The dental composite material as claimed in claim 1, wherein the adjuvant comprises 25% HDDA, 25% TPGDA and 50% EHA by weight of the adjuvant.

5. The dental composite material as claimed in claim 1, wherein the adjuvant comprises 25% HDDA, 25% TPGDA, 25% EHA and 25% IBOA by weight of the adjuvant.

6. The dental composite material as claimed in claim 1, wherein the composite resin comprises 2,2-bis-4-2-hydroxy-3-methacryloxypropoxy-propane and triethylene-glycol-dimethacrylate (TEGDMA).

7. The dental composite material as claimed in claim 1, further comprising photoactives with a concentration of less than 2% by weight of the dental composite material.

8. The dental composite material as claimed in claim 7, wherein the photoactives comprises camphorquinone (CQ), ethyl 4-dimethylaminobenzoate (EDMAB) and Benzoyl peroxide (BPO).

9. A method for restoring tooth after root canal treatment, comprising:
filling a dental composite material into a root canal, wherein the dental composite material comprises a composite resin and an adjuvant with a concentration of more than 10% by weight of the dental composite material, and the adjuvant comprises HDDA with a concentration ranging from 20% to 25% by weight of the adjuvant, TPGDA with a concentration ranging from 20% to 25% by weight of the adjuvant and a component with a concentration ranging from 20% to 50% by weight of the adjuvant; and the component is selected from the group consisting of EHA, IBOA, 2 Hydroxy 3 Phenoxypropyl Acrylate and the arbitrary compositions thereof;
disposing at least one root canal post in the root canal; and
curing the dental composite material to fasten the root canal post.

10. The method for restoring tooth as claimed in claim 9, wherein the root canal post is a cast metal post or a fiber post.
